(19) United States
(12) Patent Application Publication
(10) Pub. No.: US 2026/0283836 A1
(43) Pub. Date: Sep. 24, 2026

(54) CONVEX OSTOMY BARRIER WITH FLEXIBLE SUPPORT CONVEXITY

(71) Applicant: Hollister Incorporated, Libertyville, IL (US)

(72) Inventor: James P. Wines, Algonquin, IL (US)

(73) Assignee: Hollister Incorporated, Libertyville, IL (US)

(21) Appl. No.: 19/476,562

(22) PCT Filed: Jun. 19, 2024
(86) PCT No.: PCT/US24/34626

§ 371 Date: Oct. 17, 2025

Related U.S. Application Data
(60) Provisional application No. 63/513,257, filed on Jul. 12, 2023.

(51) Int. Cl. A61F 5/443

(52) U.S. Cl. CPC A61F 5/443

(57) ABSTRACT

A convex ostomy barrier assembly is provided. The convex ostomy barrier assembly can include a convex barrier, an inlet opening, and at least one radial convex insert. The convex barrier can include a dome area that provides a convexity. The inlet opening can be defined in the convex barrier for receiving a stoma. The at least one radial convex insert can be arranged in the dome area to provides a localized support.

### BACKGROUND

This disclosure is related to an ostomy barrier having flexible support convexity.

Ostomy pouches for collecting bodily waste are used by individuals who have had surgery such as a colostomy, ileostomy, or urostomy. An ostomy pouch may be attached to a user via an ostomy barrier, which is configured to seal against peristomal skin surfaces and protect the peristomal surfaces from exposure to stomal effluent. However, the topography of stomas and peristomal surfaces surrounding stomas varies among patients, and sealing an ostomy appliance against such different peristomal surfaces and stomas remains as an area for further improvements. For example, a stoma may protrude more or less, or may even be flush or recessed. However, the convexity of a conventional convex ostomy barrier or insert is fixed and may not work efficiently for all ostomates.

Accordingly, it is desirable to provide an improved ostomy barrier appliance.

### BRIEF SUMMARY

A flexible support convexity for ostomy barriers is provided according to various embodiments.

In one aspect, a convex ostomy barrier assembly may include a convex barrier, an inlet opening, and at least one radial convex insert. The convex barrier may include a dome area configured to provide convexity. The inlet opening may be defined in the convex barrier for receiving a stoma. The at least one radial convex insert may be arranged the dome area and configured to provide a localised convexity support in the dome area.

In an embodiment, the least one radial convex insert may include an inner flange, an outer flange and a central portion, wherein one of the at least one radial convex insert is configured to provide a localized convexity support in less than 50% of a total surface area of the dome area.

In an embodiment, convex ostomy barrier assembly may further include an adhesive layer provided on a body-side of the convex barrier configured to attach the convex ostomy barrier assembly to a user.

In some embodiments, the at least one radial convex insert may be attached to a distal surface in the dome area.

In an embodiment, the at least one radial convex insert any include two radial convex inserts arranged spaced apart from each other. In another embodiment, the at least one radial convex insert may include three radial convex inserts. In such an embodiment, the three radial convex inserts may be circumferentially evenly spaced relative to each other in the dome area.

In an embodiment, the at least one radial convex insert may include a first radial convex insert, a second radial convex insert, and a third radial convex insert, wherein the first radial convex insert may be arranged adjacent the second radial convex insert with a first space therebetween, and the second radial convex insert may be arranged adjacent the third radial convex insert with a second space therebetween. In such an embodiment, the first space and the second space may be equal to each other or may not be equal to each other.

In an embodiment, the at least one radial convex insert may include at least two radial convex inserts, wherein at least one folding area may be defined in at least one unsupported area between adjacent radial convex inserts of the at least two radial convex inserts. In such an embodiment, the at least one folding area may be configured to flex or bend in response to user's movements.

In some embodiments, each of the at least one radial convex insert may be configured to have a surface contour to match a distal surface contour of a corresponding dome area, such that the radial convex insert may be fittingly attached to the distal surface of the corresponding dome area.

In any of the foregoing embodiments, the at least one radial convex insert may be formed from a polymeric material. The at least one radial convex insert may be attached to the convex barrier via an adhesive or heat sealing.

In another aspect, a convex ostomy barrier may include a backing layer, a skin barrier layer arranged on a body-side surface of the backing layer configured to attach the convex ostomy barrier to user's skin, and at least two radial convex inserts configured to provide a localized convexity support. The at least two radial convex inserts may be arranged on a distal-side surface of backing layer spaced apart from each other.

In an embodiment, at least one folding area may be defined in at least one unsupported area between adjacent radial convex inserts of the at least two radial convex inserts. The at least one folding area may be configured to flex or bend in response to user's movements.

In an embodiment, the at least two radial convex inserts may include three radial convex inserts. The three radial convex inserts may be evenly spaced relative to each other in a dome area of the convex ostomy barrier, which may protrude toward a body-side of the convex ostomy barrier.

In an embodiment, the at least two radial convex insert may include a first radial convex insert, a second radial convex insert, and a third radial convex insert, wherein the first radial convex insert may be arranged adjacent the second radial convex insert with a first space therebetween and the second radial convex insert may be arranged adjacent the third radial convex insert with a second space therebetween. The first space and the second space may be equal to each other or may not equal to each other.

In some embodiments, the backing layer may be formed from a polymeric film, and the skin barrier layer may include a hydrocolloid adhesive. The at least two radial convex inserts may be formed from a polymeric material.

The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 is a distal-side perspective view of a convex barrier, according to an embodiment.

FIG. 2 is a distal-side view of the convex barrier of FIG. 1.

FIG. 3 is a distal-side view of the convex barrier of FIG. 1 with folding or bending areas.

FIG. 4 is a cross-sectional view of the convex barrier of FIG. 1.

FIG. 5 is a cross-sectional view of a radial convex insert of FIG. 1.

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated. The words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words “top,” “bottom,” up,“ ”down,“ front,” “back,” and the like are used for purposes of illustration and as such, are not limiting. Depending on the context, the word “if” as used herein may be interpreted as “when” or “upon” or “in response to determining.”

The present disclosure provides a convex ostomy barrier with flexible support convexity. The flexible support convexity can be provided by one or more radial convex inserts on the convex ostomy barrier. The flexible support convexity can include stoma support relative to body contours on the peristomal skin. In an embodiment, the convex ostomy barrier can be a convex skin barrier and can include one or more radial convex inserts attached to provide support in particular areas of the convex barrier according to a user's need. The radial convex inserts can be configured to provide firm support in specific areas while offering soft and flexible movement in non-supported areas of the convex ostomy barrier. The radial convex inserts can further provide improved and customized conformity according to the needs of different users. The radial convex inserts can include an inner ring-shaped portion and/or outer ring-shaped portion.

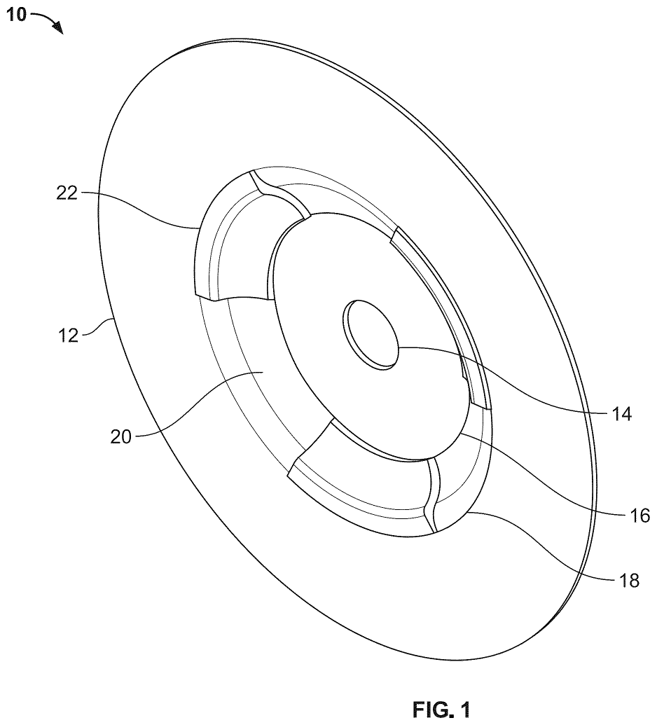

Referring now to the figures, FIGS. 1-3 show a convex skin barrier 10. The convex skin barrier 10 can be part of a convex ostomy barrier assembly. The convex skin barrier 10 can generally include a tape 12, an inlet opening 14 for receiving a stoma, an inner ring 16, an outer ring 18, a dome 20 formed between the inner and outer rings 16, 18 protruding toward a body-side, and at least one radial convex insert 22. A body-side surface of the convex skin barrier 10 can be securely mounted to a user's abdomen using the tape 12 and can provide an interface between the stoma and an ostomy pouch (not shown). The at least one radial convex insert 22 can be attached to a distal side of the convex skin barrier 10 in the dome 20 area between the inner ring 16 and outer ring 18. The at least one radial convex insert 22 can provide firm support on the section the at least one radial convex insert 22 is located in the dome 20. An ostomy pouch (not shown) can be attached to the distal side of the convex skin barrier 10 and surround the inner and outer rings 16, 18.

According to example embodiments, the tape 12 can include a first adhesive layer and a first backing layer. The first adhesive layer of the tape 12 can be formed from a suitable medical adhesive, such as an acrylic adhesive. The first backing layer can be formed from a suitable material, such as a nonwoven material or a thin polymeric film. In another embodiment, the tape 12 can include a hydrocolloid adhesive and a film backing layer.

In an embodiment, the radial convex insert 22 can be attached or mounted to a distal surface of the dome 20 using, for example, an adhesive, a heat seal, and/or can be overmolded between the hydrocolloid and a barrier backing.

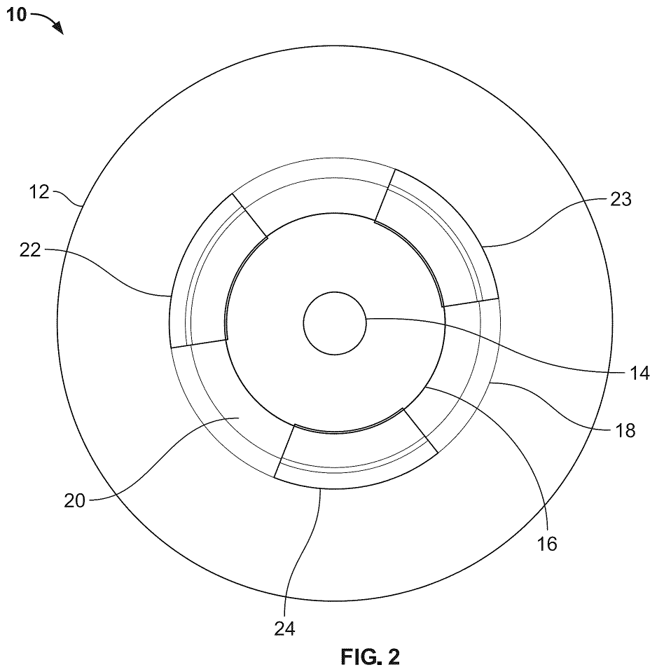

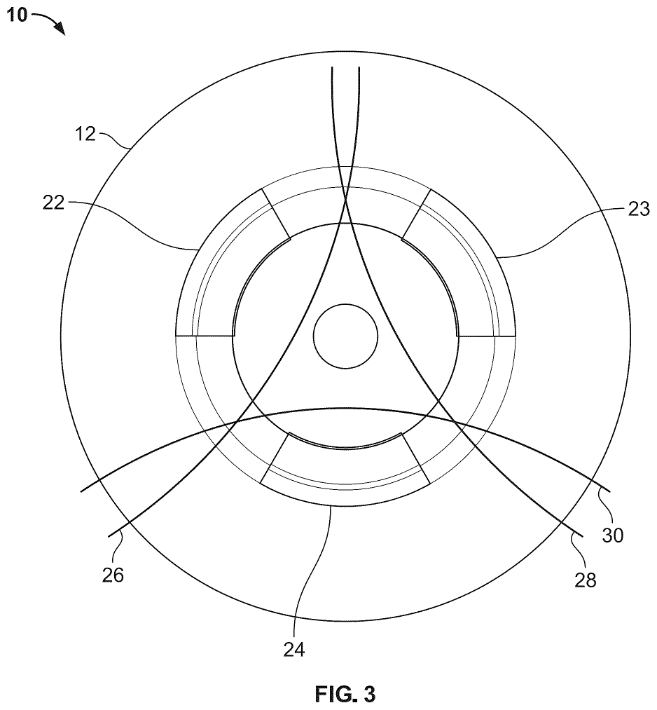

FIG. 2 illustrates an example of the convex skin barrier 10 having multiple radial convex inserts 22, 23, 24 evenly spaced on the convex skin barrier 10. FIG. 3 illustrates an example of the convex skin barrier 10 having a first, second, and third radial convex inserts 22, 23, 24. The radial convex inserts 22, 23, 24 can be evenly spaced and located in the dome 20 to provide customized and localized support to the convex skin barrier 10. For example, the radial convex inserts 22, 23, 24 can be positioned in an equally circumferentially distributed pattern to maintain stoma support with body contours. The support the radial convex inserts 22, 23, 24 provided can cause a first, second, and third folding area 26, 28, 30 between the radial convex inserts 22, 23, 24. The folding areas 26, 28, 30 can provide a freedom of movement such as a hinge or bending motion at areas where the convex skin barrier 10 can fold relative to the radial convex inserts 22, 23, 24. The support can include a convexity support at specific areas, for example, problem areas, while allowing flexing and bending at other areas and therefore alleviating pressure by the insert pushing away from the body and reducing support. This can create a firm and customized support structure while offering soft and flexible movement and an improved conformity to varying body shapes and stomas.

In an embodiment, the convex skin barrier 10 can be customized with a user's need based on the convex skin barrier's 10 orientation. For example the convex skin barrier 10 can be rotated to provide support and flexibility at specific areas. In another embodiment, the radial convex inserts 22, 23, 24 can be attached or mounted based on where support and flexibility is needed. For example, the radial convex inserts 22, 23 can be located closer to each other than to radial convex insert 24; that is the inserts need not be equally circumferentially spaced from one another.

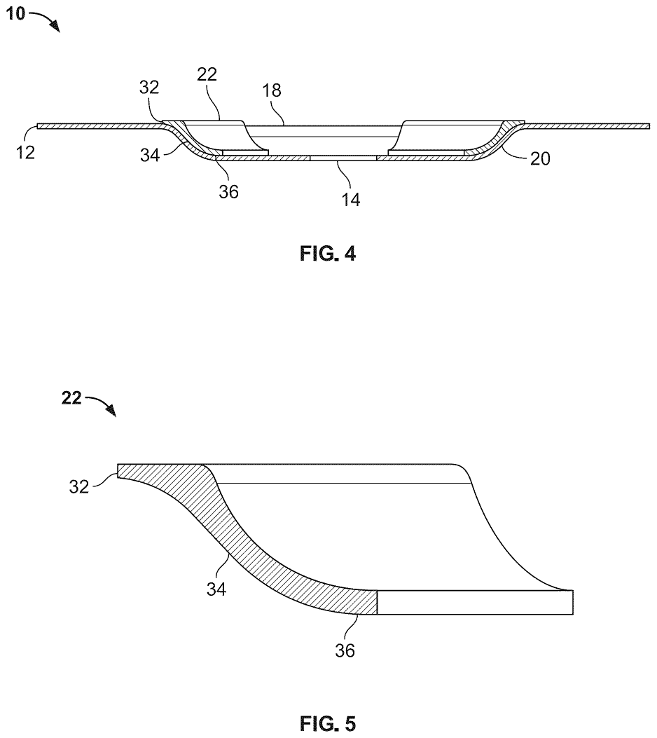

FIG. 4 illustrates a cross sectional view of an embodiment of the convex skin barrier 10. The convex skin barrier 10 can include the radial convex insert 22 with an inner flange 36, an outer flange 32, and a central portion 34. The radial convex insert 22 can be attached to the convex skin barrier 10 with the central portion 34 located along the dome 20. The radial convex insert 22 can be attached using an adhesive or heat seal such that the radial convex insert 22 is fully sealed and there is no leak path between the radial convex insert 22 and convex skin barrier 10. In an embodiment, the radial convex insert 22 can be attached to the convex skin barrier 10 during manufacturing and may be permanently fixed. In another embodiment, the radial convex insert 22 can be attached to the convex skin barrier 10 by a user or clinician before use.

In an embodiment, the radial convex inserts 22, 23, 24 can have a similar shape of that of the dome 20 to provide a secure fit that prevents affluent from collecting between the radial convex inserts 22, 23, 24 and the dome 20. The firmness and softness provided by the radial convex inserts 22, 23, 24 can be changed based on the material used to form the radial convex inserts 22, 23, 24 and/or the thickness (amount of material extending away from the dome 20) of the outer flange 32 and the central portion 34. FIG. 5 illustrates a cross sectional view of an embodiment of the radial convex insert 22. The thickness of the outer flange 32 can be larger than the thickness of the inner flange 36 to provide firm support.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A convex ostomy barrier assembly comprising:
a convex barrier, wherein the convex barrier comprises a dome area configured to provide a convexity;
an inlet opening for receiving a stoma; and
at least one radial convex insert, wherein the at least one radial convex insert is arranged in the dome area and configured to provide a localized covexity support in the dome area.

2. The convex ostomy barrier assembly of claim 1, wherein the at least one radial convex insert comprises an inner flange, an outer flange and a central portion, wherein one of the at least one radial convex insert is configured to provide a localized convexity support in less than 50% of a total surface area of the dome area.

3. The convex ostomy barrier assembly of claim 1, further including an adhesive layer provided on a body-side of the convex barrier configured to attach the convex ostomy barrier assembly to a user.

4. The convex ostomy barrier assembly of claim 1, wherein the at least one radial convex insert is attached to a distal surface in the dome area.

5. The convex ostomy barrier assembly of claim 1, wherein the at least one radial convex insert includes two radial convex inserts arranged spaced apart from each other.

6. The convex ostomy barrier assembly of claim 1, wherein the at least one radial convex insert comprises three radial convex inserts.

7. The convex ostomy barrier assembly of claim 6, wherein the three radial convex inserts are circumferentially evenly spaced relative to each other in the dome area.

8. The convex ostomy barrier assembly of claim 6, wherein the at least one radial convex insert comprise a first radial convex insert, a second radial convex insert, and a third radial convex insert, wherein the first radial convex insert is arranged adjacent the second radial convex insert with a first space therebetween, wherein the second radial convex insert is arranged adjacent the third radial convex insert with a second space therebetween, wherein the first space and the second space are not equal to each other.

9. The convex ostomy barrier assembly of claim 1, wherein the at least one radial convex insert includes at least two radial convex inserts, wherein at least one folding area is defined in at least one unsupported area between adjacent radial convex inserts of the at least two radial convex inserts, wherein the at least one folding area is configured to flex or bend in response to user's movements.

10. The convex ostomy barrier assembly of claim 1, wherein each of the at least one radial convex insert is configured to have a surface contour to match a distal surface contour of a corresponding dome area, such that the radial convex insert is fittingly attached to the distal surface of the corresponding dome area.

11. The convex ostomy barrier assembly of claim 1, wherein the at least one radial convex insert is formed from a polymeric material.

12. The convex ostomy barrier assembly of claim 1, wherein the at least one radial convex insert is attached to the convex barrier via an adhesive.

13. The convex ostomy barrier assembly of claim 1, wherein the at least one radial convex insert is heat sealed to the convex barrier.

14. A convex ostomy barrier comprising:
a backing layer;
a skin barrier layer arranged on a body-side surface of the backing layer and configured to attach the convex ostomy barrier to user's skin;
at least two radial convex inserts configured to provide a localized convexity support and arranged on a distal-side surface of backing layer spaced apart from each other.

15. The convex ostomy barrier of claim 14, wherein at least one folding area is defined in at least one unsupported area between adjacent radial convex inserts of the at least two radial convex inserts, wherein the at least one folding area is configured to flex or bend in response to user's movements.

16. The convex ostomy barrier of claim 14, wherein the at least two radial convex inserts include three radial convex inserts, wherein the three radial convex inserts are evenly spaced relative to each other in a dome area of the convex ostomy barrier, wherein the dome area protrudes toward a body-side of the convex ostomy barrier.

17. The convex ostomy barrier of claim 14, wherein the at least two radial convex insert comprise a first radial convex insert, a second radial convex insert, and a third radial convex insert, wherein the first radial convex insert is arranged adjacent the second radial convex insert with a first space therebetween, wherein the second radial convex insert is arranged adjacent the third radial convex insert with a second space therebetween, wherein the first space and the second space are not equal to each other.

18. The convex ostomy barrier of claim 14, wherein the backing layer is formed from a polymeric film and the skin barrier layer includes a hydrocolloid adhesive.

19. The convex ostomy barrier of claim 14, wherein the at least two radial convex inserts are formed from a polymeric material.
